(19) United States
(12) Patent Application Publication
(10) Pub. No.: US 2024/0390095 A1
(43) Pub. Date: Nov. 28, 2024

(54) MEDICAL DEVICE PACKAGING SYSTEMS

(71) Applicant: Anteris Technologies Corporation, Eagan, MN (US)

(72) Inventors: Tyler Kimes, Minnetonka, MN (US); Payton Kristine Parmett, Lakeville, MN (US); Michael Thomas Oswell, Andover, MN (US); Andrew E. Reed, Trumbull, CT (US); Michael R. Chmelik, Manchester, CT (US); Jesse S. Kruska, Westport, CT (US); William L. D'Agostino, Hamden, CT (US)

(21) Appl. No.: 18/669,086

(22) Filed: May 20, 2024

Related U.S. Application Data
(60) Provisional application No. 63/504,383, filed on May 25, 2023.

(51) Int. Cl. A61B 50/33; A61B 50/00; A61B 50/30

(52) U.S. Cl. CPC A61B 50/33; A61B 2050/0051; A61B 2050/006; A61B 2050/3005; A61B 2050/3007; A61B 2050/3008

(57) ABSTRACT

Packaging systems are used for medical devices. For example, this document describes packaging systems for implantable medical devices such as, but not limited to, prosthetic heart valves that are deliverable in a minimally invasive manner using a system of catheters. Some embodiments of the packaging systems described herein are designed to facilitate preparation processes that clinicians perform to prepare the medical devices for implantation.

### CROSS-REFERENCE TO RELATED APPLICATIONS

This application claims the benefit of U.S. Provisional Application Ser. No. 63/504,383 filed on May 25, 2023. The disclosure of the prior application is considered part of (and is incorporated by reference in) the disclosure of this application.

### BACKGROUND

### 1. Technical Field

This document relates to packaging systems for medical devices and methods for their use. For example, this document relates to packaging systems for implantable medical devices such as prosthetic heart valves that are deliverable in a minimally invasive manner using a system of catheters.

### 2. Background Information

Packaging systems for medical devices often serve a number of key purposes. For example, such packaging can protect the medical device contained therein from damage during shipping, storage, and handling. Such protection can help to maintain the integrity of high-value medical devices and to help ensure patient safety. Moreover, many medical devices have a requirement to be maintained in a sterile or sterilized state. Some packaging systems are especially designed to facilitate sterilization processes and to maintain thereafter the medical device in a sterile state during shipping, storage, and handling.

Some implantable medical devices require one or more preparation processes after the device is removed from packaging and before actual use. For example, some prosthetic heart valves and delivery systems must be prepped for use by medical personnel who may perform processes such as flushing of catheters, rinsing the prosthetic heart valve in solutions, and crimping or mounting the prosthetic heart valve onto a delivery catheter over an expandable balloon.

### SUMMARY

This document describes packaging systems for medical devices and methods for their use. For example, this document describes packaging systems for implantable medical devices such as, but not limited to, prosthetic heart valves that are deliverable in a minimally invasive manner using a system of catheters.

In one aspect, this disclosure is directed to a medical device system that includes an implantable device delivery system comprising: (i) a handle device and (ii) one or more catheters coupled to and extending from the handle device. The medical device system also includes a packaging system configured to contain the implantable device delivery system. The packaging system includes: (i) a first tray and (ii) a second tray releasably coupled with the handle device. The second tray is slidably coupled with the first tray.

In another aspect, this disclosure is directed to another medical device system. Such a medical device system includes an implantable device delivery system comprising: (i) a handle device and (ii) one or more catheters coupled to and extending from the handle device. The medical device system also includes a packaging system configured to contain the implantable device delivery system. The packaging system includes: (i) a first tray and (ii) a catheter support member that is releasably coupleable with the one or more catheters. The catheter support member is movable in relation to the first tray to elevate the one or more catheters relative to the first tray.

In another aspect, this disclosure is directed to another medical device system. Such a medical device system includes an implantable device delivery system comprising: (i) a handle device and (ii) one or more catheters coupled to and extending from the handle device. The medical device system also includes a packaging system configured to contain the implantable device delivery system. The packaging system includes: (i) a tray and (ii) multiple separate rinse basins releasably coupled with the tray.

In another aspect, this disclosure is directed to a medical device container. Such a medical device container includes a glass jar with a threaded open end, and a lid. The lid includes: (i) a first end portion that is releasably coupleable with the threaded open end of the glass jar and (ii) a second end portion that is affixed to the first end portion. The second end portion is at least partially transparent. An open internal space is defined between the first and second end portions.

In another aspect, this disclosure is directed to a method of preparing a prosthetic heart valve for implantation. The method includes opening a packaging system containing a medical device comprising a handle and one or more catheters extending from the handle. The package system includes a first tray, and a second tray releasably coupled with the handle device. The second tray is slidably coupled with the first tray. The method also includes, while the handle device remains coupled with the second tray, sliding the second tray relative to the first tray so that a distal end portion of the one or more catheters extends beyond the packaging system. The method also includes, while the distal end portion of the one or more catheters is extending beyond the packaging system, coupling a medical device to the distal end portion of the one or more catheters.

Particular embodiments of the subject matter described in this document can be implemented to realize one or more of the following advantages. In some embodiments, the packaging systems described herein are advantageously designed to be dual purpose, e.g., to serve as conventional packaging and, secondly, to facilitate preparation of the implantable medical device for use. For example, some embodiments of the packaging systems have special structural features that allow the implantable device delivery catheter(s) to interface with a prosthetic valve crimping device. Such features can assist medical practitioners to prepare the implantable medical device for use in a convenient, controlled, and accurate manner, and in a manner that is easier to perform than conventional processes. In some embodiments, the packaging systems described herein are advantageously designed to require less space in the medical procedure room setting. Further, some embodiments of the packaging systems described herein include other implements that are typically used during the preparation of the medical device. For example, some embodiments include a basin for receiving catheter flushing fluids, and/or separate basins for rinsing the medical device in preparation for use. Moreover, some embodiments of the packaging systems described herein advantageously include a holder for one or more other devices that are used during preparation of the medical device, such as a syringe, a balloon endo-inflator device, and the like.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1 is an exploded diagram of an example medical device packaging system in accordance with some embodiments provided herein.

FIG. 2 is an exploded diagram of a portion of the medical device packaging system of FIG. 1.

FIG. 3 illustrates a step of opening the portion of the medical device packaging of FIG. 2.

FIG. 4 illustrates another step of opening the medical device packaging of FIG. 2.

FIG. 5 illustrates a step of preparing the medical device packaging of FIG. 2 for use during an implantable medical device preparation process.

FIG. 6 illustrates a step of using the medical device packaging of FIG. 2 for preparation of an implantable medical device.

FIG. 7 illustrates another step of using the medical device packaging of FIG. 2 for preparation of the implantable medical device.

FIG. 8 illustrates another step of using the medical device packaging of FIG. 2 for preparation of the implantable medical device.

FIG. 9 illustrates an example implantable medical device container in accordance with some embodiments provided herein.

FIG. 10 is an exploded diagram of a portion of the implantable medical device container of FIG. 9.

FIG. 11 illustrates another example implantable medical device container in accordance with some embodiments provided herein.

FIG. 12 illustrates a portion of the implantable medical device container of FIG. 11.

FIG. 13 illustrates another example implantable medical device container in accordance with some embodiments provided herein.

FIG. 14 illustrates a portion of the implantable medical device container of FIG. 13.

FIG. 15 illustrates another example implantable medical device container in accordance with some embodiments provided herein.

FIG. 16 illustrates a portion of the implantable medical device container of FIG. 15.

Like reference numbers represent corresponding parts throughout.

### DETAILED DESCRIPTION

This document describes packaging systems for medical devices and methods for their use. For example, this document describes packaging systems for implantable medical devices such as, but not limited to, prosthetic heart valves that are deliverable in a minimally invasive manner using a system of catheters and a control handle. Some embodiments of the packaging systems described herein are designed to facilitate preparation processes that clinicians perform in order to prepare the medical devices for implantation.

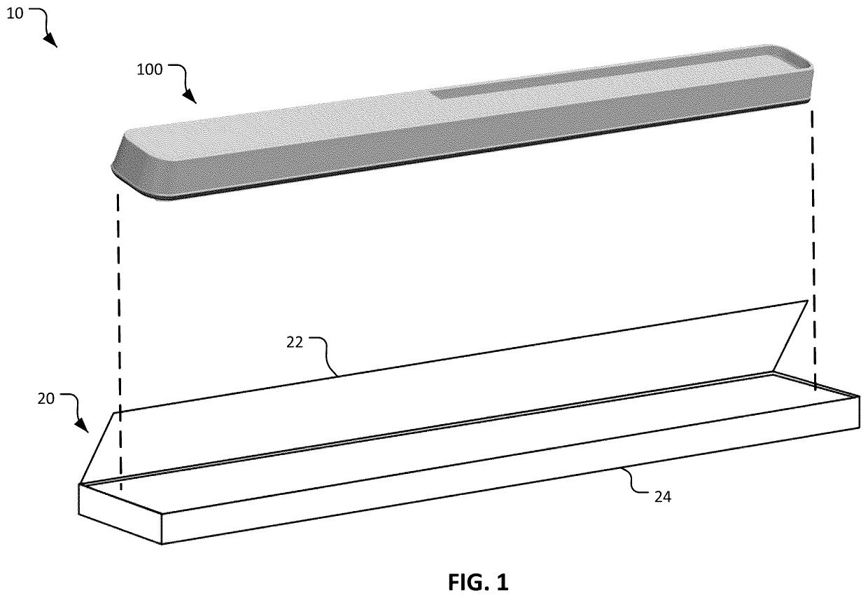

FIG. 1 is an exploded view of a medical device packaging system 10 that includes an outer box 20 and an inner packaging system 100. During shipment, the packaging system 100 is contained within the outer box 20. In some embodiments, the packaging system 100 is contained within a sealed pouch (not shown), and the combination of the packaging system 100 and the pouch are contained within the outer box 20. In some such embodiments, the pouch serves to maintain sterility of the medical device(s) contained within the packaging system 100.

In the depicted embodiment, the outer box 20 is advantageously designed to be open as shown. That is, the cover 22 of the box 20 can be pivoted open by a clinician to access and remove the inner packaging system 100. In this embodiment, one of the long edges of the cover 22 is attached to the lower portion 24 of the box 20 and is pivotable in relation to the lower portion 24 of the box 20 as shown. This design of the outer box 20 with its cover 22 that opens as shown is convenient in that the packaging system 100 can be removed therefrom within requiring the pulling/sliding of the packaging system 100 out of a small end of the box 20 (which would require a long physical space of about double the length of the box 20). Such space can be hard to come by in the congested environment of a medical procedure room.

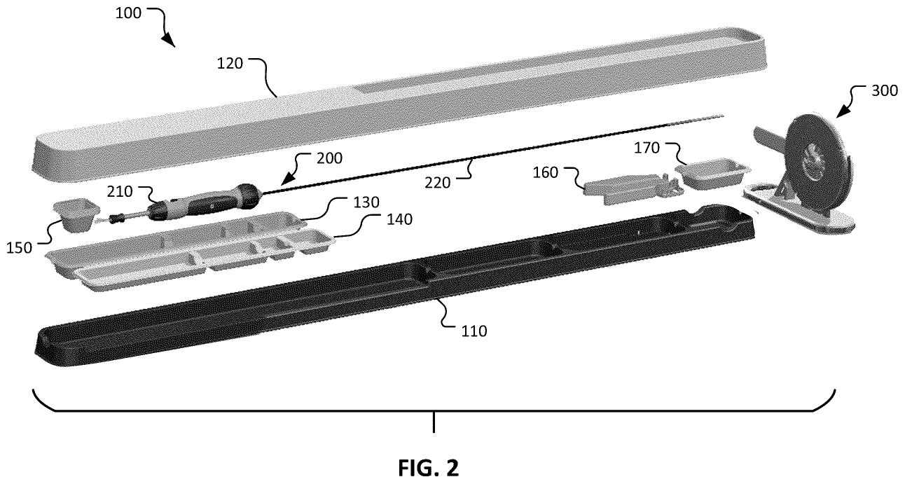

FIG. 2 shows an exploded view of the packaging system 100, an example medical device 200, and a prosthetic valve crimping device 300. In this example, the medical device 200 is packaged within the packaging system 100, but the valve crimping device 300 is not. Instead, the valve crimping device 300 is a separate device that will be used during the pre-op preparation of the medical device 200, as described further below.

The example medical device 200 includes a handle device 210 and one or more catheters 220 coupled to and extending from the handle device 210. In the depicted example, without limitation, the medical device 200 is exemplified as an implantable medical device delivery system 200, such as for a transcatheter-deliverable prosthetic heart valve (e.g., TAVR valve). It should be understood that the packaging system 100 can also be used with/for other types of medical devices.

The packaging system 100 includes a first tray 110 and a cover 120 that mates with the first tray 110 to define an internal space therein. The medical device 200 and other portions of the packaging system 100 are contained within the confines of the internal space defined between the first tray 110 and the cover 120, until unpacking is performed by a clinician.

In the depicted embodiment, the packaging system 100 also includes other useful implements that are contained within the confines of the internal space defined between the first tray 110 and the cover 120. In particular, these implements can be useful to a clinician that is preparing an implantable medical device for deployment in a patient using the implantable medical device delivery system 200. For example, in the depicted embodiment the packaging system 100 includes useful implements such as a second tray 130, a third tray 140, one or more rinse basins 150, a catheter support member 160, and a catheter flush fluid containment basin 170. Each of these implements may or may not be included in each embodiment of the packaging system 100. That is, some embodiments of the packaging system 100 may include all of the depicted implements, i.e., the second tray 130, the third tray 140, the rinse basins 150, the catheter support member 160, and the catheter flush fluid containment basin 170. Alternatively, in some embodiments, the packaging system 100 only includes particular implements of the depicted implements, i.e., one or more of: the second tray 130, the third tray 140, the rinse basins 150, the catheter support member 160, and/or the catheter flush fluid containment basin 170. Any and all such permutations/combinations of the depicted implements can be included in the various embodiments of the packaging system 100 described herein.

In the depicted embodiment, the second tray 130 is slidably coupled with the first tray 110. That is, the second tray 130 can be manually slid relative to the first tray 110 along the longitudinal direction of the first tray 110. The second tray 130 is configured to releasably receive and couple with the handle device 210. That is, the second tray 130 has structural features that are complementary with the outer profile of the handle device 210 to securing engage with the handle device 210.

In the depicted embodiment, the third tray 140 can be releasably mated with the second tray 130 (to enclose the handle device 210 therebetween). In the depicted embodiment, the third tray 140 is pivotably coupled with the second tray 130 such that the second tray 130 and the third tray 140 open like a clamshell. Alternatively, in some embodiments the third tray 140 is detachable and removable from engagement with the second tray 130. In still other embodiments, there is no third tray 140 included in the packaging system 100. The third tray 140 can be configured for releasably receiving a device such as, but not limited to, a balloon endo-inflator device.

In the depicted embodiment, the packaging system 100 also includes the one or more rinse basins 150. The one or more rinse basins 150 can be nested together with each other and removably coupled with the second tray 130 (as in the depicted embodiment), or the first tray 110, or another area of the packaging system 100.

In the depicted embodiment, the packaging system 100 also includes the catheter support member 160. The catheter support member 160 is movable relative to the first tray 110 so that the catheter support member 160 can be elevated relative to the first tray 110. In the depicted embodiment, the catheter support member 160 is pivotable relative to the first tray 110. Alternatively, the catheter support member 160 can be movable relative to the first tray 110 in other ways such as, but not limited to, telescoping it or manually inverting it 90° and sliding it into engagement with the first tray 110.

In the depicted embodiment, the packaging system 100 also includes the flush fluid containment basin 150. The flush fluid containment basin 150 can be removably coupled with the first tray 110 (as in the depicted embodiment). Alternatively, in some embodiments the flush fluid containment basin 150 is affixed or integrally formed with/in the first tray 110.

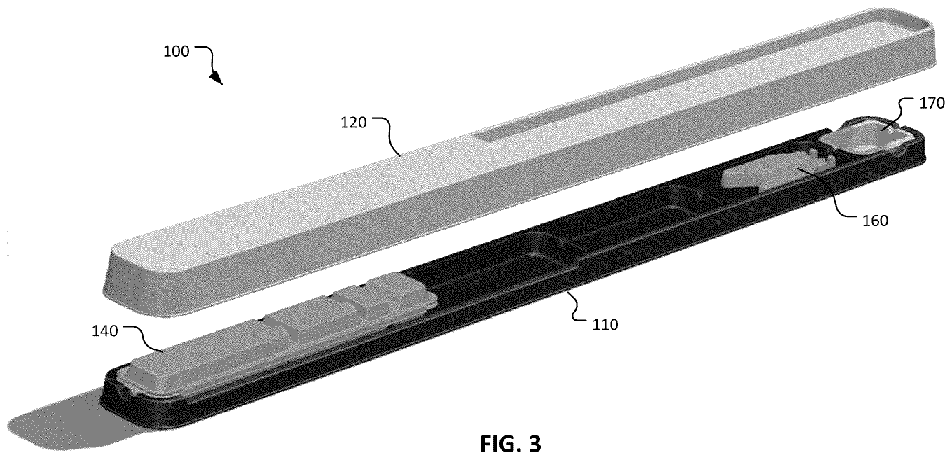

FIGS. 3-8 are a sequence of illustrations that depict the unpacking of the implantable medical device delivery system 200 from the packaging system 100, and preparation steps of the implantable medical device delivery system 200 using the packaging system 100.

FIG. 3 depicts the removal of the cover 120 from its engagement with the first tray 110. The third tray 140, the catheter support member 160, and the flush fluid containment basin 170 become visible when the cover 120 and the first tray 110 are detached from each other as shown. The medical device 200 is not shown in this illustration. The second tray 130 and the one or more rinse basins 150 are not visible in this illustration.

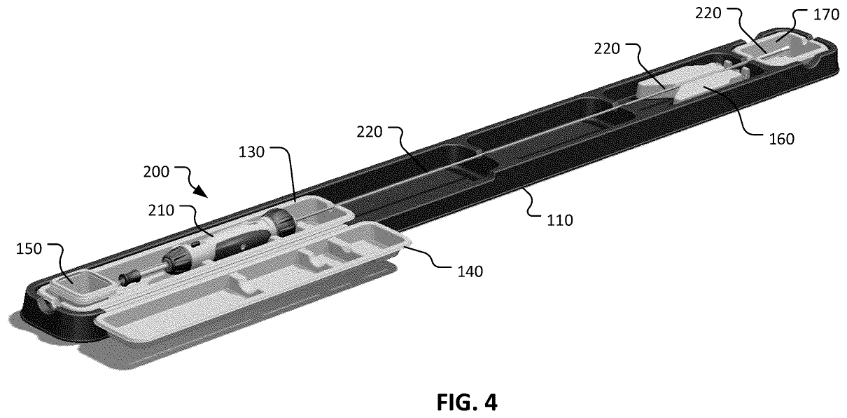

In FIG. 4, the second tray 130 and third tray 140 have been opened to reveal the implantable medical device delivery system 200 contained therebetween. The one or more rinse basins 150, which are removably coupled with the second tray 130 in this embodiment, are also revealed.

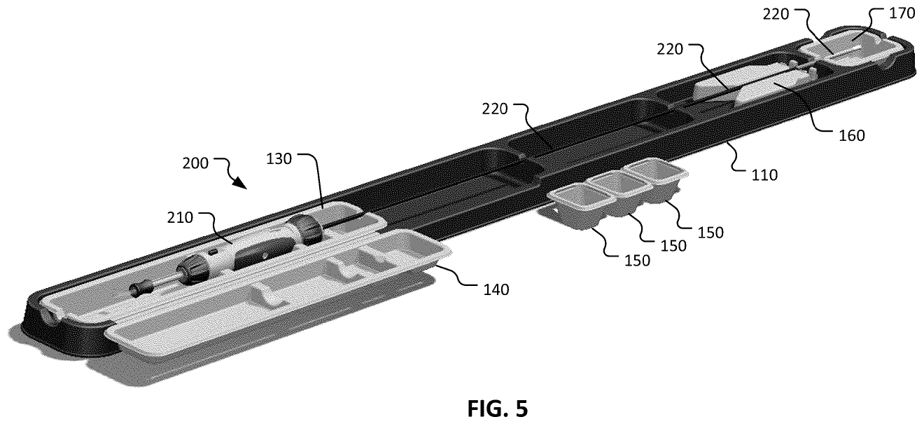

FIG. 5 shows the one or more rinse basins 150 after being removed from their home positions within the second tray 130. In the depicted embodiment, three of the rinse basins 150 are included. In some embodiments, one, two, four, five, six, seven, or more than seven of the rinse basins 150 are included in the packaging system 100.

The one or more rinse basins 150 are liquid containment vessels that can be used for various purposes including, but not limited to, for rinsing of an implantable medical device, to keep an implantable medical device hydrated, and various other purposes as desired by a medical team.

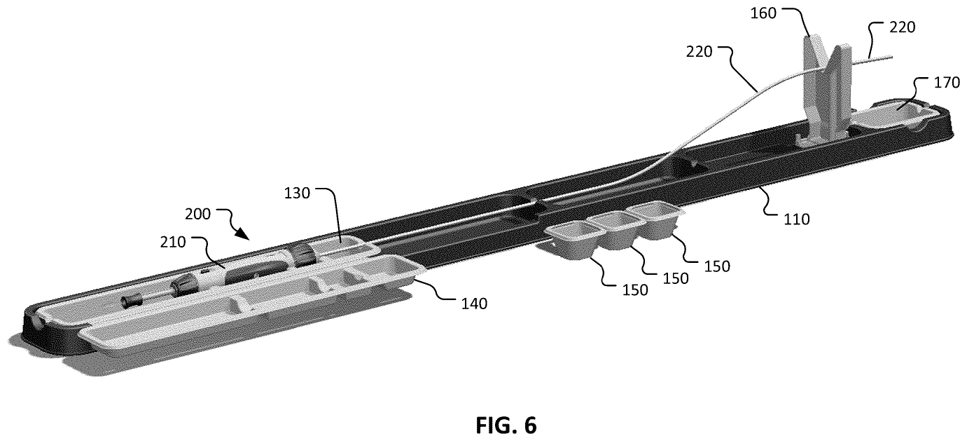

In FIG. 6, the catheter support member 160 has been manually manipulated to an elevated orientation (which, in turn, elevates the one or more catheters 220). In this elevated orientation, the catheter support member 160 extends transversely upward in relation to the first tray 110. In the depicted embodiment, a clinician can manually pivot the catheter support member 160 (e.g., by about 80° to about) 100° to the elevated orientation relative to the first tray 110 as shown (for additional clarification, compare the parallel orientation of the catheter support member 160 in FIG. 5 in which the catheter support member 160 is laying parallel with the first tray 110 with the elevated orientation of the catheter support member 160 in FIG. 6). In the depicted embodiment, the catheter support member 160 snaps into place in the elevated orientation as shown. That is, a mechanical latch serves to releasably detain the catheter support member 160 in the elevated orientation as shown.

The catheter support member 160 in its elevated orientation as shown raises a distal end portion of the one or more catheters 220. In some embodiments, the catheter support member 160 has a top surface that is shaped to releasably engage with and support the one or more catheters 220. For example, in the depicted embodiment the top surface of the catheter support member 160 has a groove in which the one or more catheters 220 rest when the catheter support member 160 is in its elevated arrangement as shown in FIG. 6. In the depicted embodiment, the groove is a V-shaped groove. The depicted elevation of the distal end portion of the one or more catheters 220 is advantageous for further preparation of the medical device 200, as described further below.

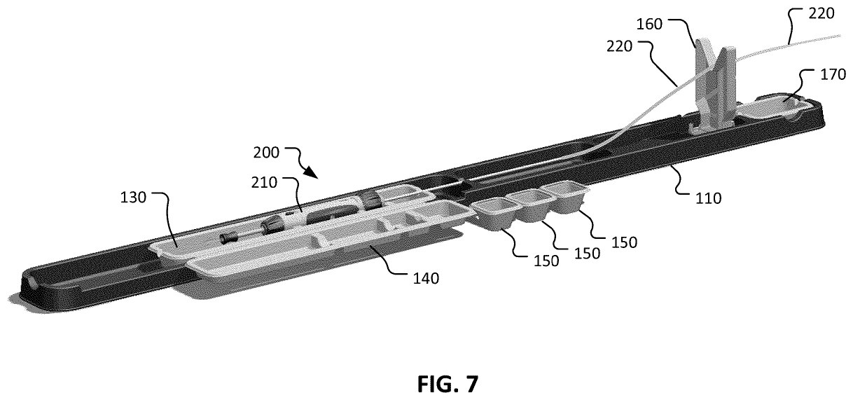

In FIG. 7, the second tray 130 has been manually slid (translated) relative to the first tray 110. That is, the second tray 130 has been manually slid along the longitudinal orientation of the first tray 110, toward the catheter support member 160. For additional clarification of this changed orientation, compare the orientation of the second tray 130 to the first tray 110 as shown in FIG. 6 with the orientation of the second tray 130 to the first tray 110 as shown in FIG. 7. This translation of the second tray 130 also translates the handle device 210 and the one or more catheters 220 because the handle device 210 is releasably coupled with the second tray 130. Accordingly, the elevated distal end portion of the one or more catheters 220 is now extending beyond the end of the first tray 110.

With the elevated distal end portion of the one or more catheters 220 now extending beyond the end of the first tray 110, the distal end portion of the one or more catheters 220 is accessible for further processing by a clinician. For example, in some cases, the valve crimping device 300 (see FIG. 2) can be used to crimp (compress) a prosthetic heart valve onto a balloon member of the distal end portion of the one or more catheters 220.

In the depicted embodiment, the third tray 140 has moved along with the second tray 130 because the two are attached to each other. However, in some embodiments the third tray 140 and the second tray 130 are not attached to each other when they are in the opened state as shown.

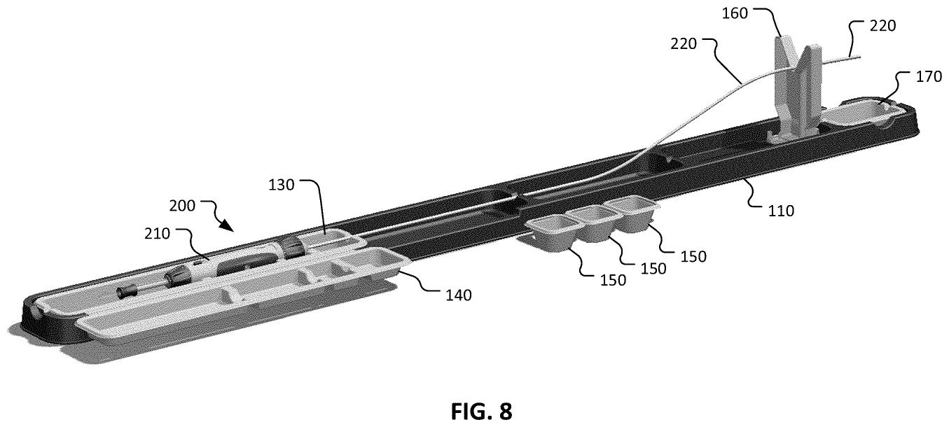

FIG. 8 shows the second tray 130 after it has been slid (translated) back to its home position relative to the first tray 110. At this point, the clinician can remove the implantable medical device delivery system 200 from the packaging system 100 and then provide it to a physician for use to treat a patient.

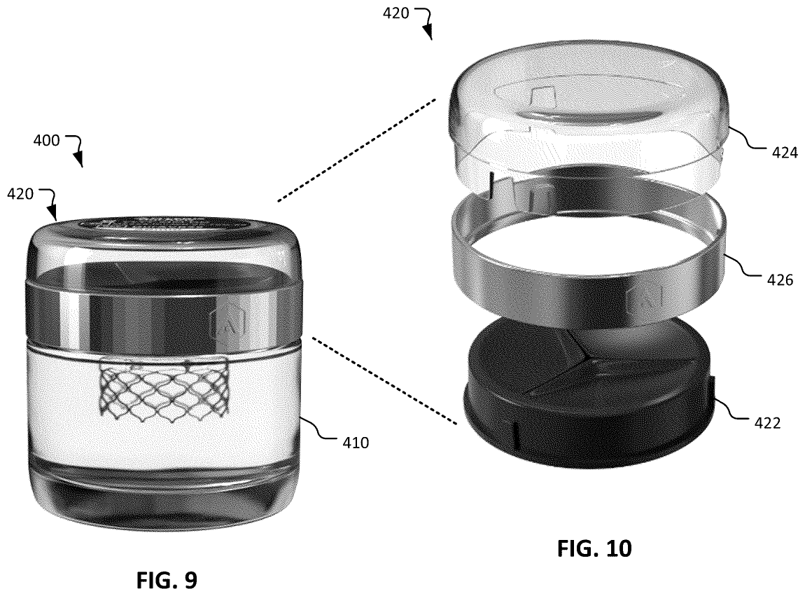

FIGS. 9-16 illustrate various embodiments of medical device containers that can be used to store and transport implantable medical devices in a liquid (e.g., propylene oxide, an aldehyde solution, a glutaraldehyde solution, a formaldehyde based solution, saline solutions, etc.). In the depicted embodiment, the example medical device is a prosthetic heart valve.

FIG. 9 shows an example medical device container 400. The medical device container 400 is configured to contain a liquid and a medical device (a prosthetic heart valve in this non-limiting example). Broadly speaking, the medical device container 400 includes a jar 410 and a lid 420. The jar 410 and the lid 420 are attachable to each other to create leak-proof internal space in which the liquid and medical device reside. The jar 410 and the lid 420 are also detachable from each other so that a clinician can access the medical device when desired.

The liquid in the medical device container 400 can serve to maintain the medical device in a desired state (e.g., where tissues (e.g., bovine pericardium or other types of tissues) of the medical device are maintained in a hydrated, stable, and biocompatible state). In the depicted embodiment, the medical device is held in a fixed position within the medical device container 400. Alternatively, in some embodiments the medical device can be allowed to move freely around within the liquid contained within the medical device container 400. The medical device container 400 can also be configured for sterilization.

In the depicted embodiment, the jar 410 is made of glass and has a threaded open end. The lid 420 has a compatible threaded end portion that can be screwed into engagement with the jar 410 to create an openable, but leak-proof connection.

FIG. 10 shows an exploded view of the lid 420. However, it should be understood that the lid 420 is constructed to be a singular, unitary member that is not intended for disassembly. The components of the lid 420 may be joined to each other using various techniques such as, but not limited to, heat staking, using adhesive, ultrasonic welding, threading, and so on, and combinations thereof.

In this non-limiting example, the lid 420 includes a first end portion 422, a second end portion 424, and an optional band 426. The first end portion 422 includes the internal threads that allow for the threaded engagement between the lid 420 and the jar 410. In some embodiments, the liquid facing side of the first end portion 422 is coated with a biocompatible material that prevents transfer of the material from which the first end portion 422 is made to the liquid. Such a coating can also serve to enhance the liquid-tight seal between the lid 420 and the jar 410.

The second end portion 424 is at least partially visually transparent, or fully visually transparent. An open internal space is defined between the first end portion 422 and the second end portion 424. Because the second end portion 424 is at least partially visually transparent, the open internal space is visually apparent and visually appealing. In some embodiments, branding or other images can be included on the side of the first end portion 422 that faces the second end portion 424, and the transparent nature of the second end portion 424 can allow a user to visualize such branding or images.

In this embodiment, the lid 420 includes the optional band 426. The band 426 can serve to help join the first end portion 422 and the second end portion 424 to each other. In addition, the band 426 can add to the visual appeal of the jar 410, and can provide a good place for branding and/or other labeling.

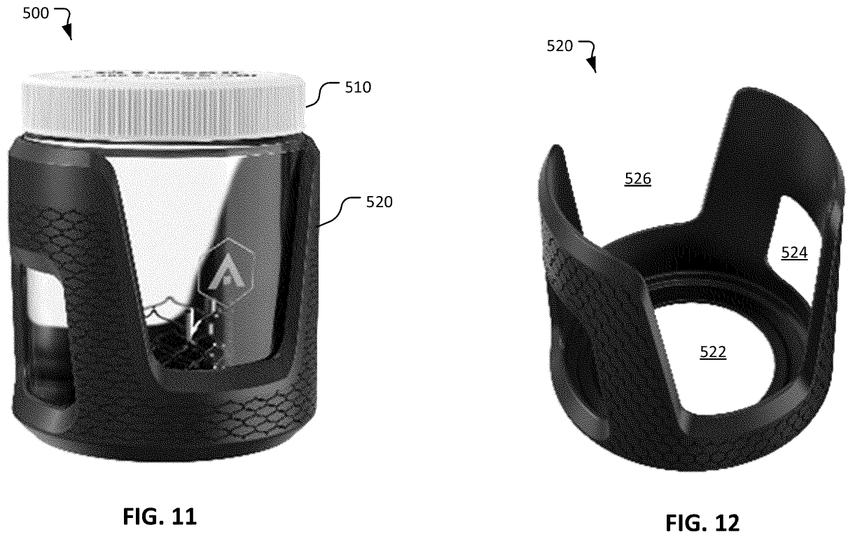

FIG. 11 illustrates another type of medical device container 500. The medical device container 500 is configured to contain a liquid and a medical device (a prosthetic heart valve in this non-limiting example). The medical device is freely movable within the liquid in this example. The jar 510 provides leak-proof internal space in which the liquid and medical device reside. The jar 510 is also openable so that a clinician can access the medical device when desired. The medical device container 500 is constructed for sterilization.

Broadly speaking, the medical device container 500 includes a jar 510 (including a lid in this example) and a sleeve 520. The sleeve 520 can be permanently attached to the jar 510 by an adhesive, for example. The sleeve 520 provides cushioning to protect the jar 510 from impacts, for example. Accordingly, the sleeve 520 can be made of a suitable resilient/compliant rubber-like material. The sleeve 520 may also provide enhanced gripping for a clinician when handling and/or opening the medical device container 500. Accordingly, in some embodiments the sleeve 520 can be manufactured with a textured outer surface.

FIG. 12 shows the sleeve 520 in isolation from the jar 510. The sleeve 520 includes a number of open portions so that the medical device is visible within the medical device container 500 (as shown in FIG. 11). For example, in this embodiment the sleeve 510 defines an open bottom 522, two windows 524, and two open side areas 526 that are unbounded at the top. These open areas can have any type of shape. In this example, the open bottom 522 is circular, the two windows 524 are quadrilaterals, and the two open side areas 526 are three-sided.

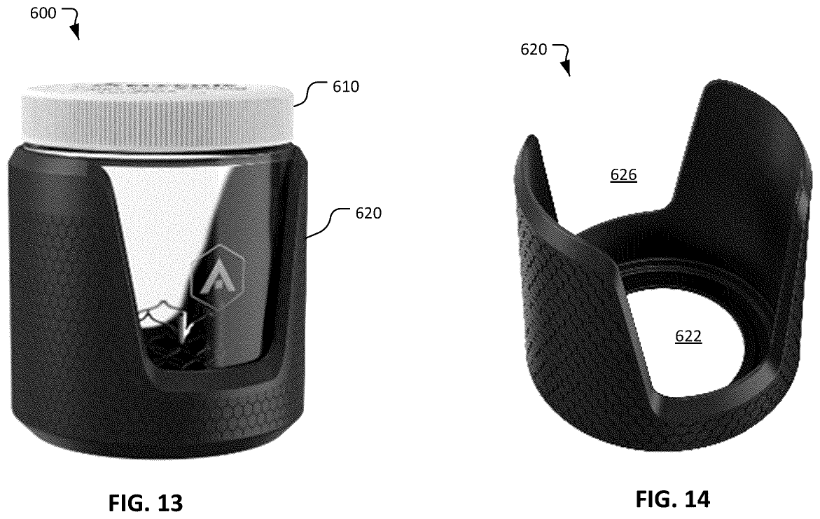

FIGS. 13 and 14 illustrate another embodiment of a medical device container 600. This is similar to the medical device container 500 except the sleeve 620 does not include the two windows. The sleeve 620 does define the open bottom 622 and the two open side areas 626 that are unbounded at the top.

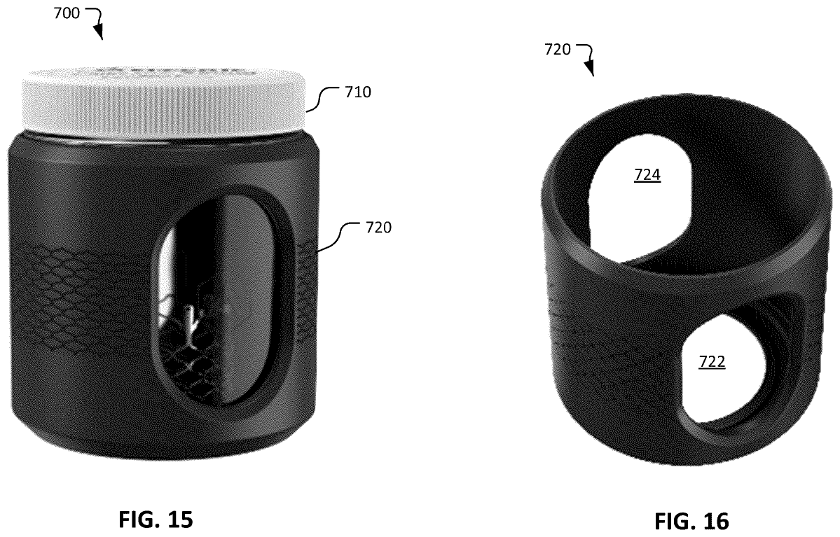

FIGS. 15 and 16 illustrate yet another embodiment of a medical device container 700. This is similar to the medical device container 500 except the sleeve 720 does not include the two open side areas that are unbounded at the top and the two windows 724 are elongated openings that are ovals in this example.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results.

What is claimed is:

1. A medical device system comprising:
an implantable device delivery system comprising: (i) a handle device and (ii) one or more catheters coupled to and extending from the handle device; and
a packaging system configured to contain the implantable device delivery system, the packaging system comprising: (i) a first tray and (ii) a second tray releasably coupled with the handle device,
wherein the second tray is slidably coupled with the first tray.

2. The system of claim 1, further comprising a catheter support member that is movably coupled to the first tray.

3. The system of claim 2, wherein the catheter support member is movable between (i) a first position in which the catheter support member is laying down parallel to the first tray and (ii) a second position in which the catheter support member is extending transversely upward out of and away from the first tray.

4. The system of claim 3, wherein the catheter support member latches in the second position.

5. The system of claim 2, wherein the catheter support member is pivotably coupled to the first tray.

6. The system of claim 2, wherein the catheter support member defines a groove to releasably support the one or more catheters.

7. The system of claim 1, further comprising a third tray that is configured to cover the second tray.

8. The system of claim 7, wherein the third tray is pivotably coupled with the second tray such that the second tray and the third tray are openable like a clamshell.

9. The system of claim 1, wherein the second tray is configured to releasably receive the handle device and is slidable along a longitudinal axis of the first tray.

10. The system of claim 1, further comprising multiple separate rinse basins that are releasably coupled with the second tray.

11. A medical device system comprising:
an implantable device delivery system comprising: (i) a handle device and (ii) one or more catheters coupled to and extending from the handle device; and
a packaging system configured to contain the implantable device delivery system, the packaging system comprising: (i) a first tray and (ii) a catheter support member that is releasably coupleable with the one or more catheters,
wherein the catheter support member is upwardly movable to an elevated orientation in relation to the first tray to elevate the one or more catheters relative to the first tray.

12. The system of claim 11, further comprising a latch mechanism by which the catheter support member is detained in the elevated orientation.

13. The system of claim 11, wherein the catheter support member is pivotably coupled to the first tray.

14. The system of claim 11, wherein the catheter support member defines a groove to releasably support the one or more catheters in the elevated orientation.

15. The system of claim 11, further comprising a second tray that releasably contains the handle device and that is longitudinally slidable along a longitudinal axis of the first tray.

16. The system of claim 15, further comprising a third tray that is configured to releasably cover the second tray.

17. A medical device system comprising:
an implantable device delivery system comprising: (i) a handle device and (ii) one or more catheters coupled to and extending from the handle device; and
a packaging system configured to contain the implantable device delivery system, the packaging system comprising: (i) a first tray, (ii) a second tray, and (iii) multiple separate rinse basins releasably coupled with the first tray or the second tray.

18. The system of claim 17, wherein the second tray releasably contains the handle device and is longitudinally slidable along a longitudinal axis of the first tray.

19. The system of claim 17, wherein the multiple separate rinse basins are nested together within the second tray.

20. The system of claim 17, further comprising a catheter support member that is upwardly movable to an elevated orientation in relation to the first tray to elevate the one or more catheters relative to the first tray.
